(19) United States
(12) Patent Application Publication
(10) Pub. No.: US 2018/0365827 A1
(43) Pub. Date: Dec. 20, 2018

(54) CREATION OF A DECISION SUPPORT MATERIAL INDICATING DAMAGE TO AN ANATOMICAL JOINT

(71) Applicant: Episurf IP-Management AB, Stockholm (SE)

(72) Inventors: Richard LILLIESTRÅLE, STOCKHOLM (SE); Anders KARLSSON, KAVLINGE (SE); Jeanette SPÅNGBERG, SKOGAS (SE); Nina BAKE, LIDINGÖ (SE); Ingrid BRATT, SOLNA (SE)

(21) Appl. No.: 16/010,344

(22) Filed: Jun. 15, 2018

Related U.S. Application Data
(63) Continuation-in-part of application No. 15/625,873, filed on Jun. 16, 2017.

(51) Int. Cl. G06T 7/00; A61B 6/00

(52) U.S. Cl. CPC G06T 7/0012; A61B 2034/108; A61B 6/5217; A61B 6/466

(57) ABSTRACT

In accordance with one or more embodiments herein, a system for creating an interactive decision support material indicating damage to at least a part of an anatomical joint of a patient is provided. The system comprises a storage media and at least one processor which is configured to: i) receive a plurality of medical image stacks of at least a part of the anatomical joint from the storage media, where each medical image stack has been generated during a scanning process using a specific sequence, wherein each specific sequence uses a unique set of parameters; ii) obtain a three-dimensional image representation of the at least part of the anatomical joint which is based on one of said medical image stacks by generating said three-dimensional image representation in an image segmentation process based on said medical image stack, or receiving said three-dimensional image representation from the storage media; iii) identify tissue parts of the anatomical joint, including at least cartilage, tendons, ligaments and/or menisci, in at least one of the plurality of medical image stacks and/or the three-dimensional image representation; iv) determine damage to the identified tissue parts in the anatomical joint by analyzing at least one of said plurality of radiology image stacks ; v) mark damage to the anatomical joint in the obtained three-dimensional image representation; vi) obtain at least one interactive 3D model based on the three-dimensional image representation in which damage has been marked; and vii) generate an interactive decision support material comprising: the at least one interactive 3D model, in which the determined damage to the at least part of the anatomical joint is marked; at least one medical image from one of the plurality of medical image stacks; and functionality to browse the medical image stack to which said medical image belongs.

### CROSS-REFERENCE TO RELATED APPLICATIONS

This application is a continuation-in-part of U.S. application Ser. No. 15/625,873, filed Jun. 16, 2017, entitled “SYSTEM AND METHOD FOR CREATING A DECISION SUPPORT MATERIAL INDICATING DAMAGE TO AN ANATOMICAL JOINT” and further incorporates by reference for all purposes the full disclosure of PCT Application No.______, filed concurrently herewith, entitled “CREATION OF A DECISION SUPPORT MATERIAL INDICATING DAMAGE TO AN ANATOMICAL JOINT” (Attorney Docket No. 0107246-002W00) and co-pending U.S. patent application Ser. No. 15/611,685, filed Jun. 1, 2017, entitled “SYSTEM AND METHOD FOR CREATING A DECISION SUPPORT MATERIAL INDICATING DAMAGE TO AN ANATOMICAL JOINT,” which is a continuation of U.S. patent application Ser. No. 15/382,523, filed Dec. 16, 2016, entitled “SYSTEM AND METHOD FOR CREATING A DECISION SUPPORT MATERIAL INDICATING DAMAGE TO AN ANATOMICAL JOINT,” which claims benefit of EP Application No. 15201361.1, filed Dec. 18, 2015, the content of which are incorporated by reference herein in their entirety.

### TECHNICAL FIELD

The present disclosure relates generally to systems and methods for creating a decision support material indicating damage to at least a part of an anatomical joint of a patient.

### BACKGROUND

In order to determine damage to an anatomical joint, it is common in medical practice today to use imaging techniques to depict the anatomical joint of interest and further to have a medical expert analyze the captured image data to determine whether there is damage. The medical expert then makes annotations about the conclusions drawn from the analysis of image data. The annotations are made available to a surgeon or orthopedic staff member who uses the annotations and the captured image data as a decision support for diagnosis and decision of suitable treatment of the patient.

However, this process is not very efficient as a manner of providing decision support, as only a fraction of the information that the medical expert in this way gathers when analyzing the image data, based on the knowledge of the medical expert, can be communicated in the present annotation format. Therefore, the decision support material received by the surgeon or orthopedic staff member is often inadequate.

Pierre Dodin et al: “A fully automated system for quantification of knee bone marrow lesions using MRI and the osteoarthritis initiative cohort”, Journal of Biomedical Graphics and Computing, 2013, Vol. 3,No. 1, 20 Nov. 2012 describes an automated bone marrow lesion (BML) quantification method.

WO 2015/117663 describes a method of manufacturing a surgical kit for cartilage repair in an articulating surface of a joint in which a three dimensional image representation of a surface of the joint is generated.

US 2014/0142643 describes a method of designing repair objects for cartilage repair in a joint, where cartilage damage to be used for the design of the repair objects is identified in image data representing a three dimensional image of a bone member of the joint.

### PROBLEMS WITH THE PRIOR ART

While the methods of the prior art may determine damage to at least bone parts of an anatomical joint, they do not provide for the creation of any type of decision support material based on the determined damage.

There is a need to address these problems of conventional methods and systems.

### SUMMARY

The above described problems are addressed by the claimed system for creating an interactive decision support material indicating damage to at least a part of an anatomical joint of a patient. The system may comprise a storage media and at least one processor which is configured to: i) receive a plurality of medical image stacks of the at least part of the anatomical joint from the storage media, where each medical image stack has been generated during a scanning process using a specific sequence, wherein each specific sequence uses a unique set of parameters; ii) obtain a three-dimensional image representation of the at least part of the anatomical joint which is based on one of said medical image stacks, by generating said three-dimensional image representation in an image segmentation process based on said medical image stack, or receiving said three-dimensional image representation from the storage media; iii) identify tissue parts of the anatomical joint in at least one of the plurality of medical image stacks and/or the three-dimensional image representation; iv) determine damage to the identified tissue parts in the anatomical joint by analyzing at least one of the plurality of medical image stacks; v) mark damage to the anatomical joint in the obtained three-dimensional image representation; vi) obtain at least one interactive 3D model based on the three-dimensional image representation in which damage has been marked; and vii) generate an interactive decision support material comprising: the at least one interactive 3D model, in which the determined damage to the at least part of the anatomical joint is marked; at least one medical image from one of the plurality of medical image stacks; and functionality to browse the medical image stack to which said medical image belongs.

In embodiments, the at least one processor is configured to use a different medical image stack for obtaining the three-dimensional image representation than each of the medical image stacks used for determining damage to the identified tissue parts in the anatomical joint.

In embodiments, the at least one processor is configured to mark the position of the displayed medical image in the interactive 3D model.

In embodiments, the at least one processor is configured to associate the medical images and the three-dimensional image representation, so that a marking made in one of the images appears in the same position in the other image. This simplifies the marking process.

The at least one processor may be configured to identify the tissue parts by e.g. detecting high contrast areas such as edges or contours in the image, and identifying structures, such as bone and/or cartilage, in the image through comparing the detected edges or contours with predefined templates.

The at least one processor may be configured to determine damage to the identified tissue parts by using a selection of: detecting an irregular shape of a contour of at least one tissue part of the anatomical joint; and/or detecting that the intensity in an area within or adjacent to bone and/or cartilage parts of the anatomical joint is higher or lower than a predetermined value; and/or comparing at least one identified tissue part with a template representing a predefined damage pattern for an anatomical joint. The claimed system creates an interactive decision support material which clearly visualizes the extent of damage to the joint or a part of the joint, such as damage to the cartilage and underlying bone, and/or damage to other tissue parts such as e.g. tendons, ligaments and/or menisci.

Each medical image stack may e.g. be captured during a process of scanning through different layers of the anatomical joint or part of it.

In embodiments, the at least one processor is configured to select a suitable treatment from a predefined set of treatments based on data from the medical image stacks and/or the three-dimensional image representation of the at least part of the anatomical joint. The treatment may e.g. be the selection of a suitable implant from a predefined set of implants with varying dimensions, or the proposal of a transfer guide tool for graft transplantation, possibly including a suitable size and/or suitable harvesting and/or implantation positions for osteochondral autograft plugs. In this case, the at least one processor may further be configured to visualize the selected implant and/or the suitable transfer guide tool and/or the suitable harvesting and/or implantation positions for at least one osteochondral autograft plug in the interactive 3D model and/or the displayed medical image.

The above described problems are also addressed by the claimed method for creating an interactive decision support material indicating damage to at least a part of an anatomical joint of a patient. The method may comprise the steps of: i) receiving a plurality of medical image stacks of the at least part of the anatomical joint, where each medical image stack has been generated during a scanning process using a specific sequence, wherein each specific sequence uses a unique set of parameters; ii) obtaining a three-dimensional image representation of the at least part of the anatomical joint which is based on one of said medical image stacks by generating said three-dimensional image representation in an image segmentation process based on said medical image stack, or receiving said three-dimensional image representation from a storage media; iii) identifying tissue parts of the anatomical joint in at least one of the plurality of medical image stacks and/or the three-dimensional image representation using image analysis; iv) determining damage to the identified tissue parts in the anatomical joint by analyzing at least one of said plurality of medical image stacks; v) marking damage to the anatomical joint in the obtained three-dimensional image representation; vi) obtaining at least one interactive 3D model based on the obtained three-dimensional image representation in which damage has been marked; and vii) generating an interactive decision support material comprising: the at least one interactive 3D model, in which the determined damage to the anatomical joint is marked; at least one medical image from one of the plurality of medical image stacks; and functionality to browse the medical image stack to which said medical image belongs. The claimed method creates an interactive decision support material which clearly visualizes the extent of damage to the joint or a part of the joint.

In embodiments, each of the medical image stacks used for determining damage to the identified tissue parts in the anatomical joint is different from the medical image stack used for obtaining the three-dimensional image representation.

The method may further comprise marking, in the interactive 3D model, the position of the displayed medical image.

The method may further comprise associating the medical images and the three-dimensional image representation so that a marking made in one of the images appears in the same position in the other image. This simplifies the marking process.

The tissue parts of the joint may be identified e.g. by the steps of detecting high contrast areas such as edges or contours in the image, and identifying structures, such as bone and/or cartilage, in the image through comparing the detected edges or contours with predefined templates.

The damage to the identified tissue parts may be determined using a selection of: detecting an irregular shape of a contour of at least one tissue part of the anatomical joint; and/or detecting that the intensity in an area within or adjacent to bone and/or cartilage parts of the anatomical joint is higher or lower than a predetermined value; and/or comparing at least one identified tissue part with a template representing a predefined damage pattern for an anatomical joint.

The method may further comprise selecting a suitable treatment from a predefined set of treatments based on data from the medical images and/or the three-dimensional image representation of the at least part of the anatomical joint. The treatment may e.g. be the selection of a suitable implant from a predefined set of implants with varying dimensions, or the proposal of a transfer guide tool for osteochondral autograft transplantation, possibly including a suitable size and/or suitable harvesting and/or implantation positions for osteochondral autograft plugs. In this case, the method may further comprise visualizing the selected implant and/or the suitable transfer guide tool and/or the suitable harvesting and/or implantation positions for at least one osteochondral autograft plug in the interactive 3D model.

In embodiments of the above described systems and methods, the functionality to browse the medical image stack comprises functionality to select a medical image in the medical image stack through interaction with the interactive 3D model.

In embodiments of the above described systems and methods, the medical images are radiology images, such as e.g. MR images or CT images.

In embodiments of the above described systems and methods, the medical images are MR images, and the scanning process is an MR scanning process using a number of specific MR sequences, where each specific MR sequence uses a unique set of MR parameters.

In embodiments of the above described systems and methods, the medical images are CT images, and the scanning process is a CT scanning process using a number of specific CT sequences, where each specific CT sequence uses a unique set of CT parameters.

In the above described systems and methods, the image segmentation process may e.g. depend on a segmentation process control parameter set. If both bone parts and cartilage parts of the anatomical joint are identified, damage may be determined to both the bone parts and the cartilage parts. The anatomical joint may be a knee, but may also be another joint such as an ankle, a hip, a toe, an elbow, a shoulder, a finger or a wrist. The interactive decision support material may e.g. be adapted to be used by medical staff. It may include a recommendation for a suitable treatment for repair of the determined damage.

The above described problems are also addressed by an interactive decision support material indicating damage to at least a part of an anatomical joint of a patient generated by the method steps of any one of the above described methods.

The above described problems are also addressed by a non-transitory machine-readable medium on which is stored machine-readable code which, when executed by a processor, controls the processor to perform any one of the above described methods.

The tissue parts of the anatomical joint may e.g. be cartilage, tendons, ligaments and/or menisci.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 shows a schematic view of a system for creating an interactive decision support material indicating damage to at least a part of an anatomical joint of a patient, in accordance with one or more embodiments described herein.

FIG. 2 is a flow diagram for a method for creating an interactive decision support material indicating damage to at least a part of an anatomical joint, in accordance with one or more embodiments described herein.

FIG. 3 shows an example of a visual representation of an interactive decision support material comprising a number of medical images and an interactive 3D model in which damage to an anatomical joint is graphically marked, in accordance with one or more embodiments described herein.

FIG. 4 shows an example of a visual representation of an interactive decision support material in which the position in the interactive 3D model of the displayed medical image is graphically marked, in accordance with one or more embodiments described herein.

FIG. 5 shows an example of a visual representation of an interactive decision support material in which type and placement of a suitable implant is indicated, in accordance with one or more embodiments described herein.

FIG. 6 is a flow diagram for a method for creating an interactive decision support material indicating damage to at least a part of an anatomical joint, in accordance with one or more embodiments described herein.

FIG. 7 is a flow diagram exemplifying the steps from obtaining medical image data to designing and producing an implant and/or guide tool for repair of a determined damage to an anatomical joint, including the steps of damage marking and generation of an interactive decision support material in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

### Introduction

The present disclosure relates generally to systems and methods for creating an interactive decision support material indicating damage to at least a part of an anatomical joint of a patient.

More specifically, system and method embodiments presented herein provide an interactive decision support material by creating at least one interactive 3D model of at least a part of an anatomical joint of a patient, in which damage to the joint or a part of the joint is marked. In other words, there is provided one or more visualizations of a patient's joint together with indications/markings/visualization of its anatomical deviations, which form a decision support for a surgeon or orthopedic staff member in deciding on an optimal treatment method, a decision support for an insurance agent making an assessment regarding a client or potential client, a decision support for a patient who wants to be informed about the condition of a damaged joint, or a decision support for any other person who has for example a commercial or academic interest in learning about damage to a depicted anatomical joint. This provides great advantages compared to conventional systems and methods, as much more information obtained from the medical image data is communicated, for example to the person making the decision on treatment of the patient. Thereby, embodiments of the invention solve the identified problems that the decision support material received by the surgeon or orthopedic staff member is many times inadequate as only a fraction of the information that a medical expert gathers when analyzing the image data, based on the knowledge of the medical expert, is communicated. In other words, using embodiments presented herein, an interactive decision support material is obtained, which leads to more informed decisions being made on the optimal treatment of the patient whose anatomical joint is depicted in the decision support material.

In some embodiments, the anatomical joint is a knee, but the methods and systems presented herein may be used for creating decision support material indicating damage to any suitable anatomical joint, e.g. an ankle, a hip, a toe, an elbow, a shoulder, a finger or a wrist. The decision support material need not relate to a whole anatomical joint—often only a part of the joint is of interest, such as e.g. the femoral part of the knee joint.

In a non-limiting example, the anatomical joint is a knee and the damage/anatomical deviations that are determined and indicated/marked/visualized in the interactive 3D model are related to the femoral part of the knee joint, such as chondral and/or osteochondral lesions. In another non-limiting example, the anatomical joint is an ankle and the damage/anatomical deviations that are determined and indicated/marked/visualized in the interactive 3D model are related to the talus.

The interactive decision support material may comprise at least one interactive 3D model of the anatomical joint and medical image data retrieved directly from a digital imaging and communications in medicine (DICOM) file or any other suitable image file format. The interactive 3D model may for example be obtained based on a medical image stack captured during a process of scanning images through different layers of the anatomical joint or part of it.

Each medical image stack may e.g. be generated during a scanning process using a specific sequence, comprising a unique set of parameters that differs from the set of parameters used for generating the other medical image stacks. Such a scanning process may be any type of scanning process for generating medical image stacks, where different sets of parameters may be used to generate medical image stacks with different types of detail. The use of different specific sequences for different uses of the medical image stacks allows the visualization of more detail in the images, since some types of detail may be more clearly visible using one set of parameters and other types of detail may be more clearly visible using another set of parameters. It may e.g. be useful to use an adapted sequence in the scanning process for generating the medical image stack used for generating the interactive 3D model, since the requirements on such a medical image stack are different from the requirements on the medical image stack used for damage determination.

The scanning processes used for generating the medical image stacks may e.g. be MR scanning processes using different specific MR sequences, where each specific MR sequence uses a unique set of MR parameters. The MR parameters may e.g. be the repetition time TR (the time between the RF pulses) and the echo time TE (the time between an RF pulse and its echo). Depending on the desired information, the set of MR parameters may e.g. cause a T1 weighted MR sequence if a short TR and a short TE is selected, a T2 weighted MR sequence if a long TR and a long TE is selected, or an intermediately weighted MR sequence of a long TR and a short TE is selected. The different sets of MR parameters do not necessarily have to cause MR sequences of different types—two different sets of MR parameters may e.g. both cause T1 weighted sequences, but one of the sets may cause a stronger T1 weighting than the other. There are also other MR parameters, such as e.g. flip angle, bandwidth or different types of fat suppression or enhancement of gadolinium, which may be varied between the MR sequences.

In MR scanning, it may be advantageous to use very different sets of MR parameters for generating the medical image stack used for generating the interactive 3D model and for generating the other medical image stacks. It may e.g. be advantageous to use a specific 3D MRI sequence for generating the medical image stack used for generating the interactive 3D model. In a 2D MRI sequence, each radiofrequency (RF) pulse excites a narrow slice, and magnetic field gradients are applied in two directions parallel to the plane in order to analyze the result. Such slices may then be combined into a 3D volume. In a 3D MRI sequence, on the other hand, each RF pulse excites the entire imaging volume, and magnetic field gradients are applied in three directions in order to analyze the result. In this way, a 3D volume may be created directly. Encoding (e.g. phase encoding) may be used to discriminate spatially.

The scanning processes used for generating the medical image stacks may also be CT scanning processes using different specific CT sequences, where each specific CT sequence uses a unique set of CT parameters. The CT parameters may e.g. be the tube potential (kV), the tube current (mA), the tube current product (mAs), the effective tube current-time product (mAs/slice), the tube current modulation (TCM), the table feed per rotation (pitch), the detector configuration, the collimation, the reconstruction algorithm, the patient positioning, the scan range and/or the reconstructed slice thickness. Also in CT scanning, it may be advantageous to use very different sets of CT parameters for generating the medical image stack used for generating the interactive 3D model and for generating the other medical image stacks.

A 3D model is advantageous for visualizing damage to bone, cartilage and other tissues. The DICOM format, or a comparable medical image file format, is advantageous for visualizing different parts of the anatomical joint. For example, a 3D model may be used for visualizing bone and tissues such as cartilage, tendons, ligaments and/or menisci, and damages in relation to femoral knee bone and cartilage, or bone and cartilage of any other relevant anatomical joint that is being investigated. In another example, the DICOM format, or a comparable medical image file format, may be used for visualizing different parts of a knee, such as the femoral condyles and the trochlea area, or different parts of any other relevant anatomical joint that is being investigated, such as the talus of the ankle.

An interactive 3D model and at least one medical image may be included in an interactive decision support material to, for instance, facilitate for a surgeon or orthopedic staff member to make a correct diagnosis and decide on an optimal treatment of the patient. The decision support material does not include any diagnosis, but instead forms a decision support for making a correct diagnosis and/or decide on an optimal treatment of the patient. The decision support material may for instance be used as a pre-arthroscopic tool, a digital version of standard arthroscopy to be used prior to an arthroscopy to give an arthroscopist a visual understanding of what he/she can expect to see. The decision support material may also be used as an alternative to arthroscopy, since enough information can often be gathered in this way without submitting the patient to an arthroscopy. The decision support material may in this case be used for planning the preferred treatment, such as an arthroplasty, a biological treatment such as a mosaicplasty of a microfracturing, or if a metal implant is needed.

In other examples, other types of users may receive and use the interactive decision support material for different purposes. The decision support material may in different situations be of interest to medical staff, an insurance agent assessing a client or a potential client, a patient who wants to be informed about the condition of a damaged joint, or any other person who has for example a commercial or academic interest in learning about damage to a depicted anatomical joint. In different embodiments, the interactive decision support material may be represented as a computer file or a web interface. A user who is viewing the decision support material on a display of a processing device may be allowed to manipulate the interactive 3D model and/or the medical image, by providing a control signal using an inputter connected to the processing device. The inputter may for example comprise a keyboard, a computer mouse, buttons, touch functionality, a joystick, or any other suitable input device.

In some embodiments, the decision support material may further include a recommendation and/or a position indication of a suitable implant for the determined bone and/or cartilage damage. In this context, a suitable implant means an implant having a type and dimensions that match a determined damage, thereby making it suitable for repairing the determined damage. Such a suitable implant may further be visualized in the interactive 3D model and/or the displayed medical image.

The interactive decision support material may in some embodiments instead include a recommendation indicating a suitable transfer guide tool and/or suitable harvesting and/or implantation positions for at least one osteochondral autograft plug. The suitable transfer guide tool and/or the suitable harvesting and implantation positions may further be visualized in the interactive 3D model and/or the displayed medical image.

In some embodiments, the decision support material further indicates anatomical deviations which do not in themselves constitute damage to the joint. Such anatomical deviations may e.g. affect the choice of treatment for the determined damage. As a non-limiting example, severe osteophyte problems may indicate other problems, where an implant may not improve the situation.

The processor may in some embodiments comprise several different processors which together perform the claimed functions. In the same way, the storage media may in some embodiments comprise several different storage media which together perform the claimed functions.

System and method embodiments of the disclosed solution are presented in more detail in connection with the figures.

### System Architecture

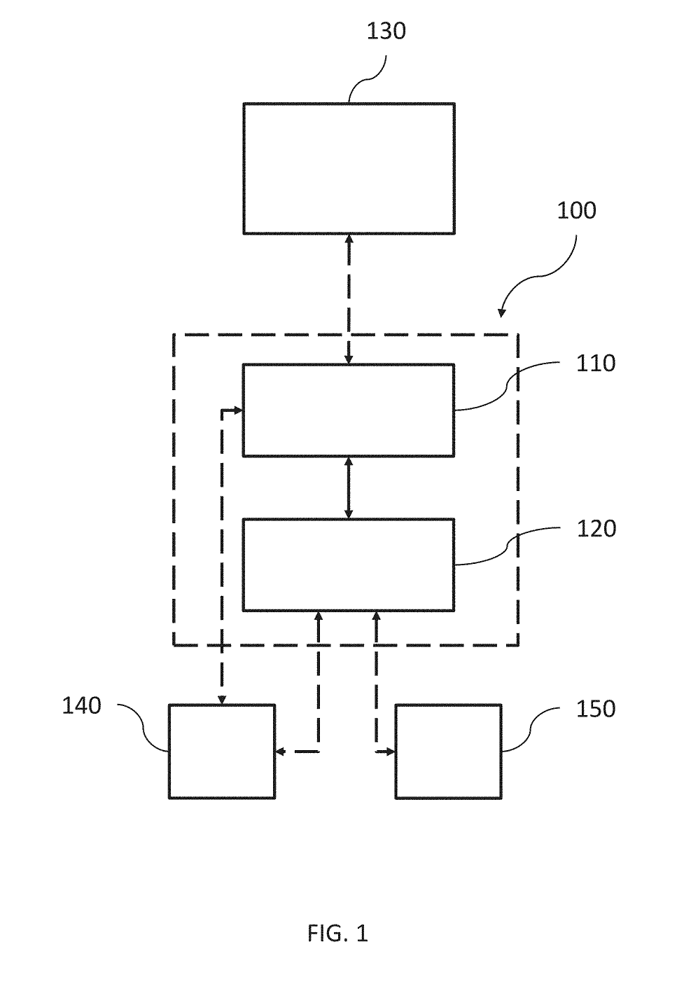

FIG. 1 shows a schematic view of a system 100 for creating an interactive decision support material indicating damage to at least a part of an anatomical joint of a patient. According to embodiments, the system comprises a storage media 110, configured to receive and store image data and parameters. In some embodiments, the system 100 is communicatively coupled, as indicated by the dashed arrow, to an imaging system 130. The imaging system 130 may be configured to capture or generate medical images, e.g. radiology images such as X-ray images, ultrasound images, computed tomography (CT) images, nuclear medicine including positron emission tomography (PET) images, and magnetic resonance imaging (MRI) images. The storage media 110 may be configured to receive and store medical images and/or medical/radiology image data from the imaging system 130.

The system 100 further comprises a processor 120 configured to, based on image data, determine damage to an anatomical joint, and create an interactive 3D model of the anatomical joint or a part of it where the determined damage to the joint is marked, or in other ways visualized, such that an observer of the interactive 3D model is made aware of the damage. The processor 120 may for example be a general data processor, or other circuit or integrated circuit capable of executing instructions to perform various processing operations.

In one or more embodiments, the processor 120 is configured to: receive a plurality of medical image stacks of the at least part of the anatomical joint from the storage media 110, where each medical image stack has been generated during a scanning process using a specific sequence, wherein each specific sequence uses a unique set of parameters; obtain a three-dimensional image representation of the at least part of the anatomical joint which is based on one of said of medical image stacks by generating said three-dimensional image representation in an image segmentation process based on said medical image stack, or receiving said three-dimensional image representation from the storage media 110; identify tissue parts of the anatomical joint in at least one of the plurality of medical image stacks and/or the three-dimensional image representation; determine damage to the identified tissue parts in the anatomical joint by analyzing at least one of said medical image stacks; mark damage to the anatomical joint in the obtained three-dimensional image representation; obtain at least one interactive 3D model based on the three-dimensional image representation in which the determined damage has been marked; and generate an interactive decision support material. The interactive decision support material may comprise the at least one interactive 3D model, in which damage to the at least part of the anatomical joint is marked; at least one medical image from one of the plurality of medical image stacks; and functionality to browse the medical image stack to which said medical image belongs.

The processor 120 may be configured to use the identified tissue parts and perform a selection of the following image analysis and processing operations:
detecting an irregular shape of a contour of at least one tissue part of the anatomical joint;
detecting that the intensity in an area within or adjacent to the bone and/or cartilage parts of the anatomical joint is higher or lower than a predetermined value; and/or
comparing at least one identified tissue part with a template representing a predefined damage pattern for an anatomical joint.

It may in some embodiments be advantageous to identify and analyze bone and cartilage of the depicted joint in the input medical/radiology image data, as the combination of the two may provide additional information, but all embodiments described herein can also be performed when other tissues of the depicted joint are identified and analyzed, alone or in combination with bone and/or cartilage.

In one or more embodiments, the processor 120 may be configured to identify tissue parts of the joint in the image by detecting high contrast areas such as edges or contours in the image. The processor 120 may further be configured to identify structures such as bone and/or cartilage in the image by comparing detected edges or contours, and/or comparing intensity levels or patterns, with predefined templates.

As disclosed above, in one or more embodiments the processor 120 may be configured to, in determining that there is damage by performing a selection of image analysis and processing operations, detect that the intensity in an area within or adjacent to the bone and/or cartilage parts of the anatomical joint is higher or lower than a predetermined threshold. Depending on the settings of the imaging device that has captured the analyzed medical image data, the analyzed image may for example represent the following substances with different intensity levels: cortical bone, fluid/liquids, cartilage, tendons, ligaments, fat/bone marrow and menisci. It is for example an indication of damage if fluid is detected where there in a healthy joint should be no fluid. If fluid is detected next to abnormalities in the cartilage, this can also be an indication of damage.

Different intensity levels in the analyzed image correspond to different signal intensity levels, and these may typically be represented by pixel/voxel values ranging from 0 to 1, or in a visual representation shown as grey scale levels from white to black. In embodiments where the pixel/voxel values range from 0 to 1, a predetermined threshold is set to a suitable value between 0 and 1, or in other words to a suitable grey scale value. In one or more embodiments the processor 120 may further, or alternatively, be configured to, in performing a selection of image analysis and processing operations, detect an irregular shape of at least one tissue part of the anatomical joint and determine whether this represents a damage to the anatomical joint. In one or more embodiments the processor 120 may further, or alternatively, be configured to, in performing a selection of image analysis and processing operations, make a comparison of an identified tissue part in a damage image with a template representing a predefined damage pattern for an anatomical joint. In some embodiments, such a determination may include comparing a detected irregular shape of the contour with a template representing a predefined damage pattern for an anatomical joint, and/or comparing a detected intensity for a certain area with a template representing a predefined damage pattern for an anatomical joint.

In one or more embodiments, the processor 120 may be configured to mark, visualize or in another way indicate a determined damage to the anatomical joint in the medical images. To mark, visualize or indicate the determined damage, the processor 120 may be configured to change the pixel/voxel value of one or more pixels/voxels on, in connection with, or surrounding a pixel/voxel identified to belong to a determined damage, such that the determined damage is visually distinguished and noticeable to a user/viewer, by performing a selection of the following:
changing the luminance/intensity values of one or more pixels/voxels identified as being located on a determined damage;
changing one or more chrominance/color values of one or more pixels/voxels identified as being located on a determined damage;
changing the luminance/intensity values of one or more pixels/voxels identified as surrounding a determined damage;
changing one or more chrominance/color values of one or more pixels/voxels identified as surrounding a determined damage; and/or
adding an annotation, symbol or other damage indicator to the image, in connection with one or more pixels/voxels identified as being located on, or surrounding, a determined damage.

In one or more embodiments, the processor 120 may be configured to mark damage to the anatomical joint in the obtained three-dimensional image representation of the anatomical joint or part of it. To mark damage, the processor 120 may be configured to change the voxel value of one or more voxels on, in connection with, or surrounding a voxel identified to belong to a determined damage, such that the determined damage is visually distinguished and noticeable to a user/viewer, by performing a selection of the following:
changing the luminance/intensity values of one or more voxels identified as being located on a determined damage;
changing one or more chrominance/color values of one or more voxels identified as being located on a determined damage;
changing the luminance/intensity values of one or more voxels identified as surrounding a determined damage;
changing one or more chrominance/color values of one or more voxels identified as surrounding a determined damage; and/or
adding an annotation, symbol or other damage indicator to the image, in connection with one or more voxels identified as being located on, or surrounding, a determined damage.

In one or more embodiments, the processor may be configured to synchronize, or associate, the medical images and the three-dimensional image representation, so that a marking made in one of the images appear in real time in the same position in the other image. The same position is hereinafter interpreted as the same position, or same location, on the anatomical joint that is depicted.

The medical image stack may for example be captured during a process of scanning through different layers of the anatomical joint or part of it. In embodiments, damage may be determined for bone parts and/or cartilage parts, and/or other tissue parts, such as e.g. tendons, ligaments and/or menisci, of the anatomical joint.

In some embodiments, the anatomical joint is a knee. In other embodiments, the anatomical joint may be any other anatomical joint suitable for damage determination using image data analysis, such as ankle, a hip, a toe, an elbow, a shoulder, a finger or a wrist.

In one or more embodiments, the processor may be configured to select a suitable treatment from a predefined set of treatments. The selection may be based on data from the medical images and/or the three-dimensional image representation of the anatomical joint or part of it.

In some embodiments, the processor may be configured to select a suitable implant from a predefined set of implants with varying dimensions. In this context, a suitable implant means an implant having a type and dimensions that match a determined damage, thereby making it suitable for repairing the determined damage. In one or more embodiments, the processor may be configured to visualize the selected implant in the interactive 3D model and/or the displayed medical image.

In some embodiments, the processor may be configured to propose a transfer guide tool for osteochondral autograft transplantation, possibly also including suitable size and/or suitable harvesting and/or implantation positions for at least one osteochondral autograft plug. In this context, a suitable harvesting position means a position where a suitable autograft plug can be harvested from the patient for repairing the determined damage.

In some embodiments, the interactive decision support material is adapted to be used by medical staff, for example a surgeon or orthopedic staff member. The decision support material may then include a recommendation for a suitable treatment for repair of at least a part of the determined damage.

Alternatively, the interactive decision support material includes a recommendation for a suitable design of one or more transfer guide tools for repair of at least a part of the determined damage with osteochondral autograft transplantation. The interactive decision support material may in this case also include a recommendation for a suitable harvesting site for such an osteochondral autograft plug. Such suitable harvesting sites and/or transfer guide tools may further be visualized in the interactive 3D model and/or the displayed medical image.

In some embodiments, the interactive decision support material is adapted to be used by an insurance agent making an assessment regarding a client or potential client, a patient who wants to be informed about the condition of a damaged joint, or any other person who has for example a commercial or academic interest in learning about damage to a depicted anatomical joint.

The decision support material may e.g. be in the form of a web interface, or in the form of one or more computer files adapted to be viewed on e.g. a tablet computer or a smart phone.

In one or more embodiments, the system 100 may optionally comprise a display 140 configured to display image data, for example in the form of an interactive decision support material comprising at least one interactive 3D model, in which damage determined to an anatomical joint is marked, at least one medical image from a medical image stack, and functionality to browse the medical image stack to which said medical image belongs. The display 140 may be configured to receive image data for display via the processor 120, and/or to retrieve image data for display directly from the storage media 110, possibly in response to a control signal received from the processor 120 or an inputter 150, which is further presented below.

In some embodiments, the system 100 may further optionally comprise one or more inputters 150 configured to receive user input. The inputter 150 is typically configured to interpret received user input and to generate control signals in response to said received user input. The display 140 and the inputter 150 may be integrated in, connected to or communicatively coupled to the system 100. The inputter 150 may for instance be configured to interpret received user input that is being input in connection with the interactive 3D model, and generate control signals in response to said received user input, to trigger display of an image or manipulation of image data being displayed, wherein the manipulations may be temporary or permanent. Such manipulations may for example include providing annotations, moving or changing an image or part of an image, changing the viewing perspective, zooming in or out, and/or any other suitable form of manipulation that enables the user to view and analyze the displayed image data in an improved manner. An inputter 150 may for example comprise a selection of a keyboard, a computer mouse, one or more buttons, touch functionality, a joystick, and/or any other suitable input device. In some embodiments, the processor 120 may be configured to receive a control signal from the inputter 150 and to process image data that is being displayed, or in other words manipulate a displayed image, in response to the received control signal.

The processor 120 may be configured to use a different medical image stack for obtaining the three-dimensional image representation than each of the medical image stacks used for determining damage to the identified tissue parts in the anatomical joint. In this way, the unique set of parameters used for generating each medical image stack can be optimized to the use of the medical image stack.

The position in the interactive 3D model of the displayed medical image may be marked in the interactive 3D model. This makes it easier for the user to determine what is shown in the displayed medical image.

The functionality to browse the medical image stack may also comprise functionality to select a medical image in the medical image stack through interaction with the interactive 3D model. This is an easy way for the user to visualize interesting parts of the joint.

The processor 120 may further be configured to perform any or all of the method steps of any or all of the embodiments presented herein.

### Method Embodiments

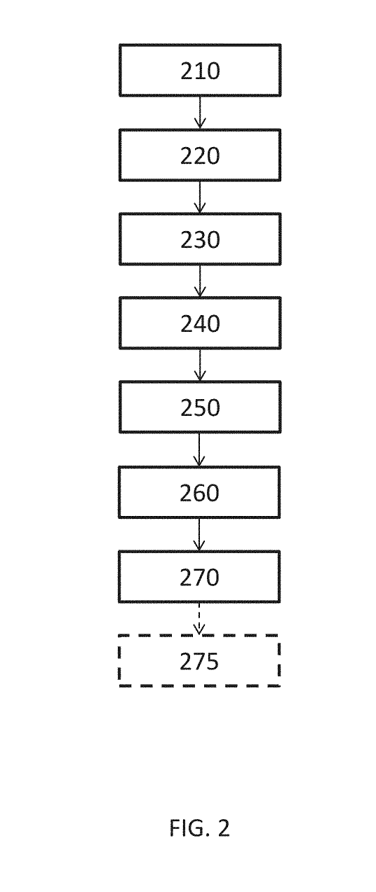

FIG. 2 is a flow diagram of method embodiments for creating an interactive decision support material indicating damage to at least a part of an anatomical joint of a patient. In accordance with one or more to embodiments, the method 200 comprises:

In step 210: receiving a plurality of medical image stacks of the at least part of the anatomical joint, where each medical image stack has been generated during a scanning process using a specific sequence, wherein each specific sequence uses a unique set of parameters.

In some embodiments, the anatomical joint is a knee. In other embodiments, the anatomical joint may be any other anatomical joint suitable for damage determination using image data analysis, such as ankle, a hip, a toe, an elbow, a shoulder, a finger or a wrist.

In step 220: obtaining a three-dimensional image representation of the at least part of the anatomical joint which is based on one of said medical image stacks by generating said three-dimensional image representation in an image segmentation process based on said medical image stack, or receiving said three-dimensional image representation from a storage media 110.

In step 230: identifying tissue parts of the anatomical joint, including at least cartilage, tendons, ligaments and/or menisci, in at least one of the plurality of medical image stacks and/or the three-dimensional image representation using image analysis.

In different embodiments, method step 230 may comprise performing a selection of any or all of the following image analysis and image processing operations:
detecting an irregular shape of a contour of at least one tissue part of the anatomical joint; and/or
detecting that the intensity in an area within or adjacent to bone and/or cartilage parts of the anatomical joint is higher or lower than a predetermined value; and/or
comparing at least one identified tissue part with a template representing a predefined damage pattern for an anatomical joint.

In one or more embodiments, tissue parts of the joint are identified in the image by the steps of detecting high contrast areas such as edges or contours in the image, and further identifying structures, such as bone and/or cartilage, in the image through comparing the detected edges or contours with predefined templates.

It may in some embodiments be advantageous to identify and analyze bone and cartilage of the depicted joint in the input medical/radiology image data, as the combination of the two may provide additional information, but all embodiments described herein can also be performed when only one of the substances bone and cartilage, and/or any other tissue part, of the depicted joint is being identified and analyzed.

In step 240: determining damage to the identified tissue parts in the anatomical joint by analyzing at least one of the plurality of medical image stacks.

In some embodiments, damage may be determined for both bone parts and cartilage parts and/or other tissue parts of the anatomical joint.

In one or more embodiments, method step 240 may comprise detecting that the intensity in an area within or adjacent to the bone and/or cartilage parts of the anatomical joint is higher or lower than a predetermined threshold. Depending on the settings of the imaging device that has captured the medical image data, the medical image may for example represent the following substances with different intensity levels: cortical bone, liquids, cartilage, tendons, ligaments, fat/bone marrow and menisci. Different intensity levels in the analyzed image correspond to different signal intensity levels and these may typically be represented by pixel/voxel values ranging from 0 to 1, or in a visual representation shown as grey scale levels from white to black. In embodiments where the pixel/voxel values range from 0 to 1, a predetermined threshold is set to a suitable value between 0 and 1, or in other words to a suitable grey scale value.

In one or more embodiments, method step 240 may further, or alternatively, comprise detecting an irregular shape of a contour of the at least one tissue part of the anatomical joint and determine whether this represents a damage to the anatomical joint.

In one or more embodiments, method step 240 may further, or alternatively, comprise making a comparison of an identified tissue part in an image with a template representing a predefined damage pattern for an anatomical joint. In some embodiments, such a determination may include comparing a detected irregular shape of the contour with a template representing a predefined damage pattern for an anatomical joint, and/or comparing a detected intensity for a certain area with a template representing a predefined damage pattern for an anatomical joint.

In step 250: marking damage to the anatomical joint in the obtained three-dimensional image representation of the anatomical joint or part of it.

In step 260: obtaining at least one interactive 3D model based on the three-dimensional image representation in which damage has been marked. The interactive 3D model may essentially correspond to the three-dimensional image representation, or be a processed version of the three-dimensional image representation.

In step 270: generating a decision support material, comprising the at least one interactive 3D model, in which damage to the anatomical joint is marked; at least one medical image from one of the plurality of medical image stacks; and functionality to browse the medical image stack to which said medical image belongs.

In embodiments, the method 200 further comprises:

In step 275: marking, in the interactive 3D model, the position of the displayed medical image.

It may in some embodiments be advantageous to identify, in step 230, and analyze, in step 240, both bone and cartilage of the depicted joint in the input medical/radiology image data, as the combination of the two may provide additional information, but all embodiments described herein may also be performed when only one of the two substances bone or cartilage, and/or any other tissue part, of the depicted joint is identified and analyzed.

In one or more embodiments, the marking of method steps 250 and 270 comprises marking, visualizing or in another way indicating the determined damage to the anatomical joint. Marking, visualizing, or indicating the determined damage may include changing the pixel/voxel value of one or more pixels/voxels on, in connection with, or surrounding a pixel/voxel identified to belong to a determined damage, such that the determined damage is visually distinguished and noticeable to a user/viewer. Such a change of pixel/voxel values of one or more pixels/voxels on, in connection with, or surrounding a pixel/voxel identified to belong to a determined damage may for example comprise a selection of the following:
changing the luminance/intensity values of one or more pixels/voxels identified as being located on a determined damage;
changing one or more chrominance/color values of one or more pixels/voxels identified as being located on a determined damage;
changing the luminance/intensity values of one or more pixels/voxels identified as surrounding a determined damage;
changing one or more chrominance/color values of one or more pixels/voxels identified as surrounding a determined damage; and/or
adding an annotation, symbol or other damage indicator to the image, in connection with one or more pixels/voxels identified as being located on, or surrounding, a determined damage.

In some embodiments, the medical image and the three-dimensional image representation may be associated, or synchronized, so that a marking made in one of the images appear in the same position in the other image. According to one or more such embodiment, the method steps may comprise associating, or synchronizing, the medical image and the three-dimensional image representation, so that a marking made in one of the images appear in the same position in the other image.

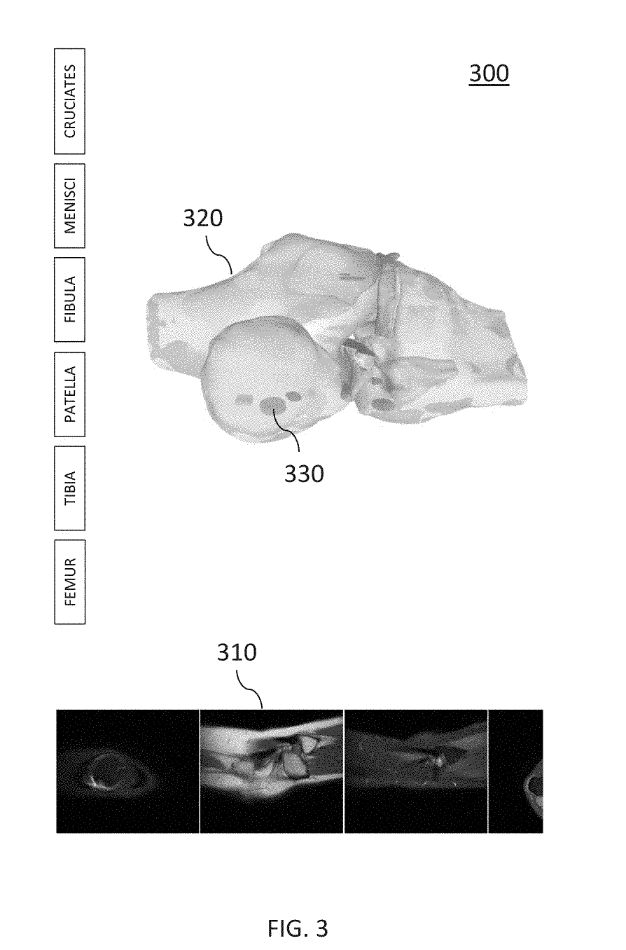

FIG. 3 shows an example of a decision support material 300 comprising a number of medical images 310 and an interactive 3D model 320 in which damage to an anatomical joint is graphically marked, in accordance with one or more embodiments described herein. In the non-limiting example shown in FIG. 3, a decision support material 300 comprises an interactive 3D model 310 of an anatomical joint, in which determined damage 330 is marked/indicated/visualized by changing the luminance/intensity levels and/or chrominance/color values of a number of pixels/voxels identified as being located on and surrounding the determined damage. Of course, any luminance/intensity values and/or chrominance/color values may be chosen, depending on the application, and depending on what provides a clear marking, visualization, or indication that enables a person viewing the decision support material to see and analyze the determined damage. A chosen luminance/intensity value and/or chrominance/color value may in embodiments be assigned to a pixel/voxel by replacing the previous pixel/voxel value, or by blending the new pixel/voxel values with the old pixel/voxel value using a scaling factor, such as an alpha blending factor. A single determined damage may further be marked, visualized, or indicated using different assigned pixel/voxel values depending on the type of damage that each pixel represents. As an example, marking, visualizing, or indicating a damage may comprise different new pixel/voxel values for:
a full-depth damage, i.e. a cartilage damage down to the bone;
a partial depth damage, such as degenerated cartilage, regenerated cartilage/scar tissue, or deformed cartilage;
a bone marrow lesion (BML); and
a distinct cyst.

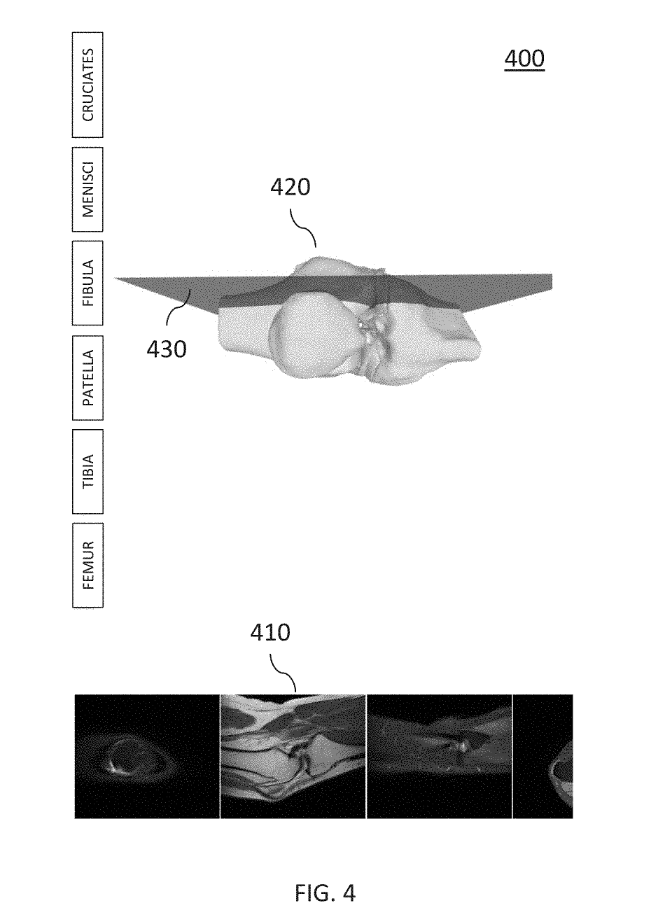

An example of how the position in the interactive 3D model of the displayed medical image may be visualized is shown in FIG. 4, which shows an example of an interactive decision support material 400 comprising a number of radiology images 410 and an interactive 3D model 420, in accordance with one or more embodiments described herein. In FIG. 4, a plane 430 in the interactive 3D model 420 shows the intersection displayed in the medical image 410. As the user browses through the medical images, the plane 430 moves in the interactive 3D model 420. The interactive decision support material 400 may also comprise functionality to select the medical images to display by indicating the desired part in the interactive 3D model 420, e.g. by moving a plane 430 through the interactive 3D model 420.

In FIGS. 3 and 4, a plurality of medical images 310, 410 are shown. The plurality of medical images 310, 410 may e.g. belong to different medical image stacks. In this way, the interactive decision support material may comprise functionality to browse through a number of different medical image stacks.

In some embodiments, the interactive decision support material may further include a recommendation and/or a position indication of a suitable implant for the determined bone and/or cartilage damage. Such a suitable implant may further be visualized in the interactive 3D model and/or the displayed medical image.

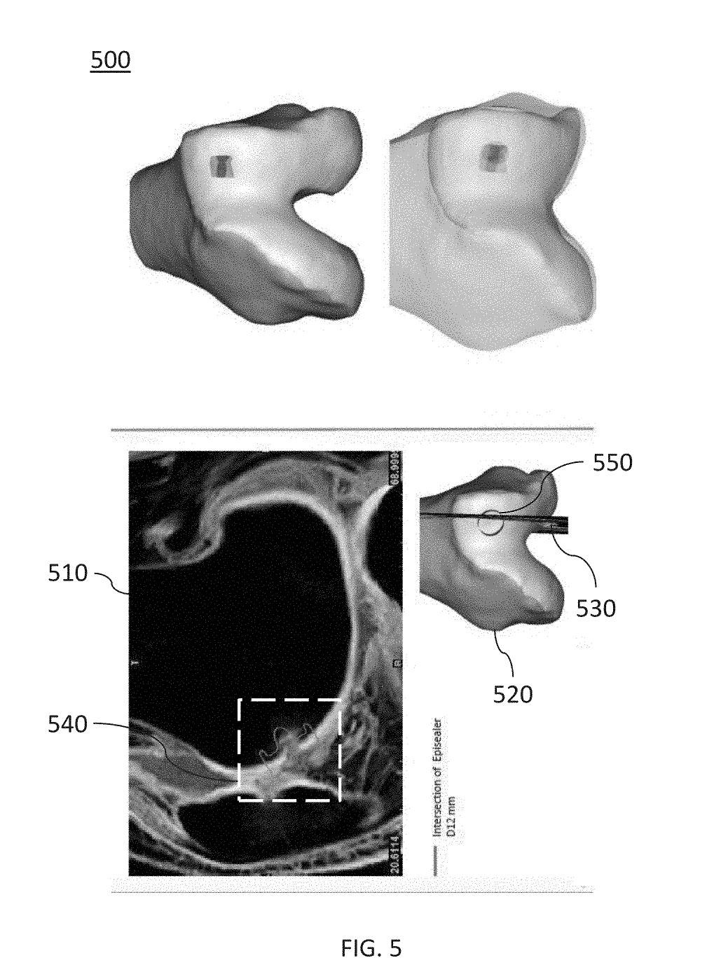

An example of how or a type and placement of a suitable implant may be indicated in the interactive decision support material is shown in FIG. 5, which comprises an interactive 3D model 520, shown in the lower part of the FIG. next to a medical image 510. In FIG. 5, a plane 530 in the interactive 3D model 520 shows the intersection displayed in the medical image 510. The type and placement of a suitable implant 540, 550 is in FIG. 5 indicated both in the interactive 3D model 520 and in the medical image 510, but it may be indicated in just the interactive 3D model. In the non-limiting example of FIG. 5, the depicted anatomical joint is a knee, and the patient has a lesion in the patella.

In one or more embodiments, the interactive decision support material is adapted to be used by medical staff, for example a surgeon or orthopedic staff member. In one or more embodiments, the interactive decision support material is adapted to be used by medical staff, for example a surgeon or orthopedic staff member, and may further include a recommendation for a suitable implant, according to any of the embodiments described above.

In some embodiments, the interactive decision support material is adapted to be used by an insurance agent making an assessment regarding a client or potential client, a patient who wants to be informed about the condition of a damaged joint, or any other person who has for example a commercial or academic interest in learning about damage to a depicted anatomical joint.

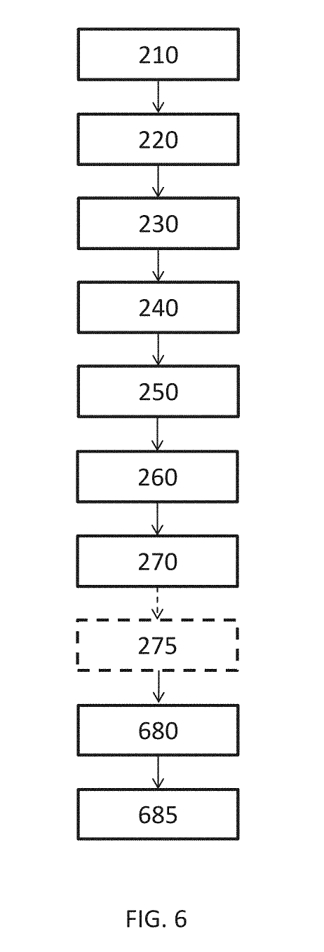

FIG. 6 is a flow diagram of one or more method embodiments for creating a damage image of an anatomical joint where damage to the joint is marked in the damage image, and further the optional method steps of including in the image a recommendation of a suitable implant for repairing a determined damage. Steps 210-275 of FIG. 6 correspond to the same steps of FIG. 2, and the method embodiments of FIG. 6 further comprise the following additional steps:

In step 680: selecting a suitable implant from a predefined set of implants with varying dimensions, based on data from the medical image and/or the three-dimensional image representation of the anatomical joint or part of it.

In this context, a suitable implant means an implant having a type and dimensions that match a determined damage, thereby making it suitable for repairing the determined damage.

In step 685: visualizing the selected implant in the interactive 3D model.

In one or more embodiments, the methods of FIGS. 2 and 6 may optionally comprise displaying a visual representation of a decision support material in a graphical user interface (GUI). The method may in any of these embodiments comprise receiving image data for display, and/or receiving a control signal and retrieving image data for display in response to the control signal.

In one or more embodiments, the interactive decision support material may be manipulated by a user using one or more inputters integrated in, connected to, or communicatively coupled to the display or a system comprising the display. According to these embodiments, the method of FIG. 2 or 6 may further optionally comprise receiving user input from an inputter, interpret the received user input, and generate one or more control signals in response to the received user input. The received user input may e.g. relate to the interactive 3D model, and generate control signals in response to said received user input to manipulate what is being displayed, temporarily or permanently. The manipulation may for example include providing annotations, moving or changing an image or part of an image, changing the viewing perspective, zooming in or out, and/or any other suitable form of manipulation that enables the user to view and analyze the displayed image data in an improved manner. In some embodiments, the method of FIG. 2 or 6 may comprise receiving a control signal from an inputter and processing the image data that is being displayed, or in other words manipulate the displayed image, in response to the control signal.

Each of the medical image stacks used for determining damage to the identified tissue parts in the anatomical joint may be different from the medical image stack used for obtaining the three-dimensional image representation. In this way, the unique set of parameters used for generating each medical image stack can be optimized to the use of the medical image stack.

The method may further comprise marking, in the interactive 3D model, the position of the displayed medical image. This makes it easier for the user to determine what is shown in the displayed medical image.

The functionality to browse the medical image stack may also comprise functionality to select a medical image in the medical image stack through interaction with the interactive 3D model. This is an easy way for the user to visualize interesting parts of the joint.

Any or all of the method steps of any or all of the embodiments presented herein may be performed automatically, e.g. by at least one processor.

### Use Case Embodiment

To set the presently disclosed methods and systems in a larger context, the damage marking and the generation of the interactive decision support material according to any of the disclosed embodiments may in use case embodiments be preceded by capturing and/or obtaining medical image data representing an anatomical joint or part of it, and may further be followed by actions to be taken in view of repairing any determined damage.

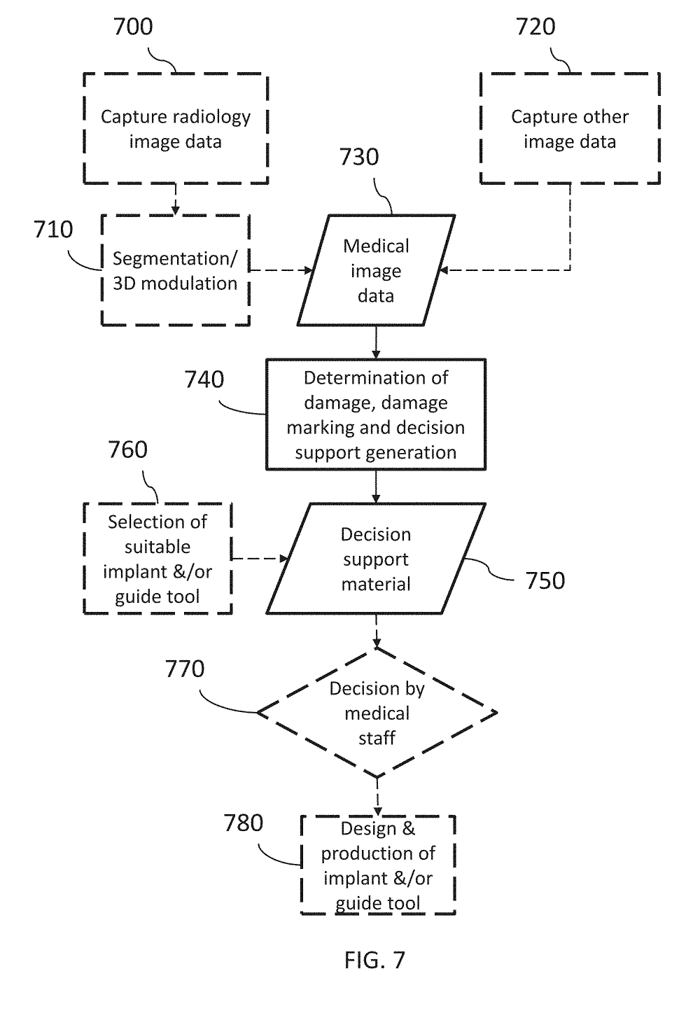

FIG. 7 is a flow diagram exemplifying one such larger context, including obtaining medical image data from an image source, determining damage to a depicted anatomical joint, and generating an interactive decision support material in accordance with one or more embodiments described herein. FIG. 7 further includes steps of designing and producing an implant and/or guide tool suitable for repairing a determined damage in an anatomical joint. In FIG. 7, everything except the determination of damage, damage marking and decision support material generation of step 740, using the input medical image data 730 and resulting in the output decision support material 750, is marked with dashed lines to clarify they are optional steps shown in the FIG. to provide context only, and not essential to any of the embodiments presented herein. Especially, steps 770 and 780 relating to diagnosis/decision on treatment and design and production of implant/guide tool are not part of the embodiments presented herein.

According to the example shown in FIG. 7, medical image data 730 may be obtained in a step 700 in the form of medical image data from a medical imaging system. The medical image data obtained may for example be radiology data, generated using one or more of a variety of medical imaging techniques such as X-ray images, ultrasound images, computed tomography (CT) images, nuclear medicine including positron emission tomography (PET) images, and magnetic resonance imaging (MRI) images. The medical image data may e.g. be captured during a process of scanning images through different layers of the anatomical joint or part of it.

Each medical image stack may e.g. have been generated during a scanning process using a specific sequence, where each specific sequence uses a unique set of parameters. Such a scanning process may be any type of scanning process for generating a series of radiology images where different sets of parameters may be used to generate images with different types of detail. The use of more than sequence allows the visualization of more detail in the image, since some types of detail may be more clearly visible using one set of parameters and other types of detail may be more clearly visible using another set of parameters.

The scanning processes used for generating the medical image stacks may e.g. be MR scanning process using different specific MR sequences, where each MR sequence uses a unique set of MR parameters. The MR parameters may e.g. be the repetition time TR (the time between the RF pulses) and the echo time TE (the time between an RF pulse and its echo). Depending on the desired information, the set of MR parameters may e.g. cause a T1 weighted MR sequence if a short TR and a short TE is selected, a T2 weighted MR sequence if a long TR and a long TE is selected, or an intermediately weighted MR sequence of a long TR and a short TE is selected. The different sets of MR parameters do not necessarily have to cause MR sequences of different types—two different sets of MR parameters may e.g. both cause T1 weighted sequences, but one of the sets may cause a stronger T1 weighting than the other. There are also other MR parameters, such as e.g. flip angle, bandwidth or different types of fat suppression or enhancement of gadolinium, which may be varied between the MR sequences. It may be advantageous to use very different sets of MR parameters for generating the medical image stack used for generating the interactive 3D model and for generating the other medical image stacks. It may e.g. be advantageous to use a specific 3D MRI sequence for generating the medical image stack used for generating the interactive 3D model.

The scanning processes used for generating the medical image stacks may also be CT scanning processes using different specific CT sequences, where each CT sequence uses a unique set of CT parameters. The CT parameters may e.g. be the tube potential (kV), the tube current (mA), the tube current product (mAs), the effective tube current-time product (mAs/slice), the tube current modulation (TCM), the table feed per rotation (pitch), the detector configuration, the collimation, the reconstruction algorithm, the patient positioning, the scan range and/or the reconstructed slice thickness. Also in CT scanning, it may be advantageous to use very different sets of CT parameters for generating the medical image stack used for generating the interactive 3D model and for generating the other medical image stacks.

The image data obtained in step 700 may further be processed in a step 710, by performing segmentation and 3D modulation to obtain a three-dimensional image representation of what is depicted in the captured image data. For instance, if the image data captured depict an anatomical joint, the three-dimensional image representation would be a three-dimensional image representation of the anatomical joint. Medical images may also be obtained in a step 720 from a different kind of image source that provides medical images. The three-dimensional image representation and the medical images both depict the same object, namely the anatomical joint of interest for damage determination. The medical image data 730 may therefore, as described herein, comprise a three-dimensional image representation and/or medical images representing an anatomical joint. The medical image data 730 may represent only a part of the anatomical joint.

The three-dimensional image representation and the medical images may in embodiments be associated, or synchronized, such that a position on an object depicted in the three-dimensional image representation is associated with the same position on the same object in the medical images. Thereby, if a marking of a determined damage is done in the three-dimensional image representation, it will appear in the same position on the depicted anatomical joint in the medical images, and vice versa. Of course, once the three-dimensional image representation and the medical images have been associated, or synchronized, the same would apply to for example annotations placed in connection with a position of the depicted joint, or any modification done to the three-dimensional image representation or the medical images.

In a step 740, damage determination, marking of damage in the input medical image data 730 and generation of the output decision support material 750 is performed, in accordance with any of the embodiments presented herein in connection with the method and system descriptions. The interactive decision support material 750 may, in accordance with embodiments described herein, comprise at least one interactive 3D model, in which damage determined to an anatomical joint is marked, at least one medical image from a medical image stack, and functionality to browse the medical image stack to which said medical image belongs. The decision support material 750 may optionally, in accordance with embodiments described herein, comprise an indication of one or more suitable implants and/or guide tools that may be used for repairing a determined damage. In this context, a suitable implant and/or guide tool means an implant and/or guide tool having a type and dimensions that match the determined damage, thereby making it suitable for repairing the determined damage. The one or more suitable implants and/or guide tools may be selected in the optional step 760, and may be presented graphically in connection with the interactive 3D model and/or the medical images of the interactive decision support material 750, for example in the position where the implant and/or guide tool should optimally be inserted to repair the determined damage. Alternatively, the one or more suitable implants and/or guide tools may be selected in the optional step 760 and may be presented separated from the interactive 3D model and/or the medical images, for example as a graphical representation and/or a text annotation.

In a use case embodiment, a medical staff member, for example a surgeon or orthopedic staff member, may use a generated interactive decision support material 750 to make a correct diagnosis and make a decision 770 on an decision of optimal treatment of the patient whose anatomical joint has been depicted. If the medical staff member decides that an implant is required, this may lead up to the step 780 of designing and producing a suitable implant and/or guide tool, possible according to an indication that may be provided in the decision support material, as described herein, for repairing the determined damage.

In another use case embodiment, a person using the interactive decision support material 750 may be a person other than a medical staff member that has an interest in learning about any damage to the depicted anatomical joint, for example an insurance agent assessing a client or a potential client, a patient who wants to be informed about the condition of a damaged joint, or any other person who has for example a commercial or academic an interest in learning about any damage to a depicted anatomical joint.

### Further Embodiments

Where applicable, various embodiments provided by the present disclosure can be implemented using hardware, software, or combinations of hardware and software. Also where applicable, the various hardware components and/or software components set forth herein can be combined into composite components comprising software, hardware, and/or both without departing from the claimed scope of the present disclosure. Where applicable, the various hardware components and/or software components set forth herein can be separated into sub-components comprising software, hardware, or both without departing from the claimed scope of the present disclosure. In addition, where applicable, it is contemplated that software components can be implemented as hardware components, and vice-versa. The method steps of one or more embodiments described herein may be performed automatically, by any suitable processing unit, or one or more steps may be performed manually. Where applicable, the ordering of various steps described herein can be changed, combined into composite steps, and/or separated into sub-steps to provide features described herein.

Software in accordance with the present disclosure, such as program code and/or data, can be stored in non-transitory form on one or more machine-readable mediums. It is also contemplated that software identified herein can be implemented using one or more general purpose or specific purpose computers and/or computer systems, networked and/or otherwise.

In embodiments, there are provided a computer program product comprising computer readable code configured to, when executed in a processor, perform any or all of the method steps described herein. In some embodiments, there are provided a non-transitory computer readable memory on which is stored computer readable and computer executable code configured to, when executed in a processor, perform any or all of the method steps described herein.

In one or more embodiments, there is provided a non-transitory machine-readable medium on which is stored machine-readable code which, when executed by a processor, controls the processor to perform the method of any or all of the method embodiments presented herein.

The foregoing disclosure is not intended to limit the present invention to the precise forms or particular fields of use disclosed. It is contemplated that various alternate embodiments and/or modifications to the present invention, whether explicitly described or implied herein, are possible in light of the disclosure. Accordingly, the scope of the invention is defined only by the claims.

1. A system for creating an interactive decision support material indicating damage to at least a part of an anatomical joint of a patient, the system comprising a storage media and at least one processor, wherein the at least one processor is configured to:
i) receive a plurality of medical image stacks of the at least part of the anatomical joint from the storage media, where each medical image stack has been generated during a scanning process using a specific sequence, wherein each specific sequence uses a unique set of parameters;
ii) obtain a three-dimensional image representation of the at least part of the anatomical joint which is based on one of said medical image stacks, by generating said three-dimensional image representation in an image segmentation process based on said medical image stack, or receiving said three-dimensional image representation from the storage media;
iii) identify tissue parts of the anatomical joint, including at least cartilage, tendons, ligaments and/or menisci, in at least one of the plurality of medical image stacks and/or the three-dimensional image representation;
iv) determine damage to the identified tissue parts in the anatomical joint by analyzing at least one of said plurality of medical image stacks;
v) mark damage to the anatomical joint in the obtained three-dimensional image representation;
vi) obtain at least one interactive 3D model based on the three-dimensional image representation in which damage has been marked; and
vii) generate an interactive decision support material comprising:
the at least one interactive 3D model, in which the determined damage to the at least part of the anatomical joint is marked;
at least one medical image from one of the plurality of medical image stacks; and
functionality to browse the medical image stack to which said medical image belongs.

2. The system according to claim 1, wherein the at least one processor is configured to use a different medical image stack for obtaining the three-dimensional image representation than each of the medical image stacks used for determining damage to the identified tissue parts in the anatomical joint.

3. The system according to claim 1, wherein the functionality to browse the medical image stack comprises functionality to select a medical image in the medical image stack through interaction with the interactive 3D model.

4. The system according to claim 1, wherein the at least one processor is configured to mark the position of the displayed medical image in the interactive 3D model.

5. The system according to claim 1, wherein the at least one processor is further configured to associate the medical images and the three-dimensional image representation, so that a marking made in one of the images appears in the same position in the other image.

6. The system according to claim 1, wherein the at least one processor is configured to identify said tissue parts by:
detecting high contrast areas such as edges or contours in the image; and
identifying structures, such as bone and/or cartilage, in the image through comparing the detected edges or contours with predefined templates.

7. The system according to claim 1, wherein the at least one processor is configured to determine damage to said identified tissue parts by using a selection of:
detecting an irregular shape of a contour of at least one tissue part of the anatomical joint; and/or
detecting that the intensity in an area within or adjacent to bone and/or cartilage parts of the anatomical joint is higher or lower than a predetermined value; and/or
comparing at least one identified tissue part with a template representing a predefined damage pattern for an anatomical joint.

8. The system according to claim 1, wherein the three-dimensional image representation is generated in an image segmentation process which depends on a segmentation process control parameter set.

9. The system according to claim 1, wherein the at least one processor is further configured to:
select a suitable implant from a predefined set of implants with varying dimensions, and/or propose a transfer guide tool for osteochondral autograft transplantation, possibly including a suitable size and/or suitable harvesting and/or implantation positions for at least one osteochondral autograft plug; and to
visualize the selected implant and/or the transfer guide tool and/or the suitable harvesting and/or implantation positions for at least one osteochondral autograft plug in the interactive 3D model.

10. A method for creating an interactive decision support material indicating damage to at least a part of an anatomical joint of a patient, the method comprising the steps of:
i) receiving a plurality of medical image stacks of the at least part of the anatomical joint, where each medical image stack has been generated during a scanning process using a specific sequence, wherein each specific sequence uses a unique set of parameters;
ii) obtaining a three-dimensional image representation of the at least part of the anatomical joint which is based on one of said medical image stacks, by generating said three-dimensional image representation in an image segmentation process based on said medical image stack, or receiving said three-dimensional image representation from a storage media;
iii) identifying tissue parts of the anatomical joint, including at least cartilage, tendons, ligaments and/or menisci, in at least one of the plurality of medical image stacks and/or the three-dimensional image representation using image analysis;
iv) determining damage to the identified tissue parts in the anatomical joint by analyzing at least one of said plurality of medical image stacks;
v) marking damage to the anatomical joint in the obtained three-dimensional image representation;
vi) obtaining at least one interactive 3D model based on the three-dimensional image representation in which damage has been marked; and
vii) generating an interactive decision support material comprising:
the at least one interactive 3D model, in which the determined damage to the anatomical joint is marked;
at least one medical image from one of the plurality of medical image stacks; and
functionality to browse the medical image stack to which said medical image belongs.

11. The method according to claim 10, wherein each of the medical image stacks used for determining damage to the identified tissue parts in the anatomical joint is different from the medical image stack used for obtaining the three-dimensional image representation.

12. The method according to claim 10, wherein the functionality to browse the medical image stack comprises functionality to select a medical image in the medical image stack through interaction with the interactive 3D model.

13. The method according to claim 10, further comprising marking, in the interactive 3D model, the position of the displayed medical image.

14. The method according to claim 10, further comprising associating the medical images and the three-dimensional image representation, so that a marking made in one of the images appears in the same position in the other image.

15. The method according to claim 10, wherein said tissue parts are identified by the steps of:
detecting high contrast areas such as edges or contours in the image; and
identifying structures, such as bone and/or cartilage, in the image through comparing the detected edges or contours with predefined templates.

16. The method according to claim 10, wherein the damage to said identified tissue parts is determined using a selection of:
detecting an irregular shape of a contour of the at least one tissue part of the anatomical joint; and/or
detecting that the intensity in an area within or adjacent to bone and/or cartilage parts of the anatomical joint is higher or lower than a predetermined value; and/or
comparing at least one identified tissue part with a template representing a predefined damage pattern for an anatomical joint.

17. The method according to claim 10, wherein the three-dimensional image representation is generated in an image segmentation process which depends on a segmentation process control parameter set.

18. The method according to claim 10, further comprising:
selecting a suitable implant from a predefined set of implants with varying dimensions, and/or proposing a transfer guide tool for osteochondral autograft transplantation, possibly including a suitable size and/or suitable harvesting and/or implantation positions for at least one osteochondral autograft plug; and
visualizing the selected implant and/or the suitable transfer guide tool and/or the suitable harvesting and/or implantation positions for at least one osteochondral autograft plug in the interactive 3D model.

19. An interactive decision support material indicating damage to at least a part of an anatomical joint of a patient generated by the method steps claim 10.
